(19) United States
(12) Patent Application Publication
(10) Pub. No.: US 2009/0220618 A1
(43) Pub. Date: Sep. 3, 2009

(54) PHARMACEUTICAL FORMULATIONS COMPRISING POLYANIONIC MATERIALS AND SOURCE OF HYDROGEN PEROXIDE

(76) Inventors: Erning Xia, Penfield, NY (US); Michael E. Allen, Webster, NY (US)

Correspondence Address: Bausch & Lomb Incorporated, One Bausch & Lomb Place, Rochester, NY, 14604-2701, US

(21) Appl. No.: 12/370,706

(22) Filed: Feb. 13, 2009

Related U.S. Application Data
(60) Provisional application No. 61/032,579, filed on Feb. 29, 2008.

(51) Int. Cl. A01N 59/00; A61P 27/02; A01P 1/00; A61K 49/00; A61K 33/40

(52) U.S. Cl. 424/616

(57) ABSTRACT

A pharmaceutical formulation that is effective in adversely affecting the viability of microorganisms or in inhibiting their growth and that provides better safety and/or comfort to the users comprises at least a polyanionic material and at least a source of hydrogen peroxide. The formulation can comprise an ophthalmically active agent for treating or controlling a disease or disorder of the eye.

### CROSS REFERENCE

This application claims the benefit of Provisional Patent Application No. 61/032,579 filed Feb. 29, 2008, which is incorporated by reference herein.

### BACKGROUND

The present invention relates to pharmaceutical formulations comprising polyanionic materials and a source of hydrogen peroxide. In particular, the present invention relates to such formulations that are used in ophthalmic applications and provide improved safety and/or comfort to the users.

Pharmaceutical formulations are commonly provided in multi-use bottles. Formulations, such as ophthalmic solutions, find uses in many ophthalmic applications. These solutions are often instilled directly into the eye one or more times a day to either deliver medications or to relieve symptoms of eye conditions, such as dry eye or inflammation of the superficial tissues of the eye accompanying various allergic reactions (such as hay fever allergies and the like, irritation of the eye due to foreign bodies, or eye fatigue). Other ophthalmic solutions are employed in the field of contact-lens care. Contact-lens solutions are utilized to soak, disinfect, clean, and wet contact lenses. These solutions are not instilled directly in the eye from the bottle, but do subsequently come into contact with the eye when the lenses are inserted.

Ophthalmic solutions are provided sterile, but once opened, are susceptible to microbial contamination. In the case of multi-use solutions, the formulations contain at least a preservative designed to kill microorganisms that come in contact with the solution, protecting the patient from infection due to a contaminated ophthalmic solution during the prescribed usage.

Typically, preservatives for ophthalmic solutions fall into two traditional categories: alcohols and amines or ammonium-containing compounds. Typical alcohol-based anti-microbial agents include benzyl alcohol, phenethyl alcohol, and chlorbutanol. Alcohol-based preservatives work by disorganizing the lipid structure of cell membrane, and thus increase permeability of the cell wall, leading to cell lysis. These alcohols have limited solubility in aqueous solutions and tend not to be stable preservatives due to being susceptible to oxidation, evaporation, and interaction with the plastic bottle. More commonly, organic amines and ammonium-containing compounds are utilized as anti-microbial agents in ophthalmic solutions. Representative compounds in this category include benzalkonium chloride (“BAK”), benzododecinium bromide (“BDD”), chlorhexidine, polymeric biguanide (such as polyhexamethylene biguanide or “PHMB”). It is believed that the electrophilicity of the nitrogen-containing moieties of these compounds promotes their interaction with the negatively charged cell membranes of the microorganisms, leading to cell lysis, and thus severely impacting their survival.

Although amines and ammonium-containing compounds have good anti-microbial activity, and are used commercially to preserve ophthalmic solutions, there are significant disadvantages associated with these compounds. In particular, these compounds used at higher doses can be toxic to the sensitive tissues of the eye. For example, BAK-containing ophthalmic solutions are known to cause eye irritation in patients. It causes growth arrest at very low concentration (0.00001%), apoptosis at 0.01%, and necrosis at higher concentrations (0.05-0.1%). Patients who may be at greater risk of BAK-induced adverse effects are those with dry-eye syndrome since they often need to use eye drop over an extended period of time. Polymeric amines and ammonium-containing compounds are less toxic than BAK but still can cause irritation responses in some other patients. For example, polyquaternium-1 (α-4-{tris(2hydroxyethyl)ammonium-2-butenyl}poly{1-dimethylammonium-2-butenyl}-ω-tris(2-hydroxyethyl)ammonium chloride), also known as Polyquad®, has been shown to be less toxic than BAK and used in a limited number of ophthalmic formulations. However, polyquaternium-1 still shows some adverse effects on ocular tissues. A 0.5% polyquaternium-1 formulation has been shown significantly to decrease goblet cell density. Healthy goblet cells are required to produce adequate mucin, which is one of three component layers of the tear film. A. Labbé et al., J. Ocular Pharmacol. & Therapeutics, Vol. 22, No. 4, 267 (2006). Chlorhexidine, on the other hand, has proven to be more biocompatible than the other amines and ammonium-containing anti-microbial agents and, therefore, non-irritating at the levels typically used. However, the mildness of chlorhexidine to the ocular environment is offset by the fact that chlorhexidine is a relatively weak preservative.

Oxidative preservatives, which work by oxidizing cell walls or membranes, affecting membrane-bound enzymes, and disrupting cellular function. U.S. Pat. Nos. 5,576,028; 5,607,698; 5,725,887; and 5,807,585 and European Patent 035486 disclose solutions, which may be ophthalmic solutions or contact lens solutions, containing from 10 ppm (0.001%) to 1000 ppm (0.1%) hydrogen peroxide and a hydrogen peroxide stabilizer. However, the long-term preservative efficacy of these solutions is not known. It is suggested that hydrogen peroxide concentration should be in trace amounts in order to be tolerable to the patient upon direct application. At trace concentrations, stabilizers are needed to prevent decomposition of hydrogen peroxide.

Therefore, there is a continued need to provide improved pharmaceutical formulations that are effective in killing microorganisms or in inhibiting their growth and that provide improved safety and/or comfort to the users. It is also very desirable to provide improved ophthalmic solutions having such advantages.

### SUMMARY

In general, the present invention provides improved pharmaceutical formulations that are effective in adversely affecting the viability of microorganisms or in inhibiting their growth and that provide better safety and/or comfort to the users.

In general, a pharmaceutical formulation of the present invention comprises at least a polyanionic material and at least a source of hydrogen peroxide.

In one aspect, such a pharmaceutical formulation is an ophthalmic solution, which provides less irritation to ocular tissues and more lubricity to ocular surfaces than prior-art solutions.

In another aspect, said at least a source of hydrogen peroxide is present in an effective amount to inhibit or prevent the survival of microorganisms.

In still another aspect, representatives of such microorganisms comprise Staphylococcus aureus, Pseudomonas aeruginosa, Eschrechia coli, Candida albicans, and Aspergillus niger.

In still another aspect, a formulation of the present invention further comprises boric acid.

In yet another aspect, a pharmaceutical formulation of the present invention is free of a material selected from the group consisting of cationic organic nitrogen-containing compounds, alcohols, and mixtures thereof.

In a further aspect, the present invention provides a method for making a pharmaceutical formulation. The method comprises combining at least a polyanionic material and at least a source of hydrogen peroxide in the pharmaceutical formulation.

In still another aspect, the present invention provides a method for providing safety, or comfort, or both to users of pharmaceutical formulation. The method comprises adding at least a polyanionic material and at least a source of hydrogen peroxide to the pharmaceutical formulation.

In yet another aspect, the present invention provides a method for treating, controlling, or preventing a condition of an eye that manifests irritation or inflammation. The method comprises topically administering to the eye an effective amount of an ophthalmic solution that comprises at least a polyanionic material and at least a source of hydrogen peroxide to relieve such irritation or inflammation.

In a further aspect, the present invention provides a method for treating an ophthalmic device. The method comprises contacting the ophthalmic device with an ophthalmic solution comprising at least a polyanionic material and at least a source of hydrogen peroxide.

In still a further aspect, the ophthalmic device is a contact lens.

Other features and advantages of the present invention will become apparent from the following detailed description and claims.

### DETAILED DESCRIPTION

In general, the present invention provides improved pharmaceutical formulations that are effective in adversely affecting the viability of microorganisms or in inhibiting their growth and that provide improved safety and/or comfort to the users, methods of making, and methods of using such formulations. Within the scope of the present invention, the microorganisms that are adversely affected by a formulation of the present invention include microorganisms selected from the group consisting of bacteria, yeasts, molds, and mixtures thereof.

In one aspect, pharmaceutical formulations of the present invention can kill or adversely affect the survival or propagation of such microorganisms. In one embodiment, representatives of such microorganisms comprise Staphylococcus aureus (or S. aureus), Pseudomonas aeruginosa (or P. aeruginosa), Eschrechia coli (or E. coli), Candida albicans (or C. albicans), and Aspergillus niger (or A. niger).

In another aspect, a pharmaceutical formulation of the present invention comprises at least a polyanionic material and at least a source of hydrogen peroxide. The term “polyanionic material,” as used herein, means a material a molecule of which comprises a plurality of negatively charged moieties and carries a net negative charge. In one embodiment, the pharmaceutical formulation comprises an ophthalmic solution.

In still another aspect, an ophthalmic solution of the present invention provides less irritation to ocular tissues and more lubricity to ocular surfaces than prior-art solutions.

In yet another aspect, said at least a source of hydrogen peroxide is present in an effective amount to inhibit or prevent the survival of microorganisms. In one embodiment, the effectiveness of the solution is determined according to a testing procedure disclosed below.

In one embodiment, said at least a source of hydrogen peroxide comprises a compound or material that release hydrogen peroxide into the formulation. In another embodiment, such a compound or material is selected from the group consisting of hydrogen peroxide, urea hydrogen peroxide (carbamide peroxide, carbamide perhydrate, or percarbamide), perborate salts, derivatives thereof, combinations thereof, and mixtures thereof.

In another embodiment, said at least a source of hydrogen peroxide is present in an amount effective to adversely affect the viability of microorganisms or inhibit their growth. In still another embodiment, said amount is effective to reduce the concentration of viable bacteria, recovered per milliliter of the formulation, at the fourteenth day after challenge, by not less than 3 logs, and after a rechallenge at the fourteenth day, said amount is also effective to reduce the concentration of viable bacteria, recovered per milliliter of the formulation, at the twenty-eighth day, by not less than 3 logs. In addition, said amount is effective to keep the concentration of viable yeasts and molds, recovered per milliliter of the formulation, at or below the initial concentration (within an experimental uncertainty of ±0.5 log) at the fourteenth day, and after a rechallenge at the fourteenth day, said amount is also effective to keep the concentration of viable yeasts and molds, recovered per milliliter of the formulation, at or below the initial concentration (within an experimental uncertainty of ±0.5 log) at the twenty-eighth day.

In still another embodiment, the amount of hydrogen peroxide generated in a pharmaceutical formulation of the present invention is in the range from about 0.0001 to about 5 percent by weight of the formulation. Alternatively, the amount of hydrogen peroxide is in the range from about 0.001 to about 3 percent, or from about 0.001 to about 1 percent, or from greater than about 0.01 to about 2 percent, or from greater than about 0.01 to about 1 percent, or from greater than about 0.01 to about 0.7 percent, or from greater than about 0.01 to about 0.5 percent, or from greater than about 0.01 to about 0.2 percent, or from greater than about 0.01 to about 0.1 percent, or from greater than about 0.01 to about 0.07 percent, or from greater than about 0.01 to about 0.05 percent, or from greater than about 0.05 to about 0.15 percent, or from greater than about 0.03 to about 0.15 percent by weight of the solution, or from greater than about 0.1 to about 1 percent, or from greater than about 0.1 to about 0.7 percent, or from greater than about 0.1 to about 0.5 percent, or from greater than about 0.1 to about 0.2 percent, or from greater than about 0.1 to about 0.15 percent. Preferably, the amount of hydrogen peroxide in a formulation of the present invention throughout its shelf life is greater than about 0.01% by weight of the total formulation.

In yet another embodiment, the amount of hydrogen peroxide generated in a pharmaceutical formulation of the present invention is in the range from 0.0001 to 5 percent by weight of the formulation. Alternatively, the amount of hydrogen peroxide is in the range from 0.001 to 3 percent, or from 0.001 to 1 percent, or from greater than 0.01 to 2 percent, or from greater than 0.01 to 1 percent, or from greater than 0.01 to 0.7 percent, or from greater than 0.01 to 0.5 percent, or from greater than 0.01 to 0.2 percent, or from greater than 0.01 to 0.1 percent, or from greater than 0.01 to 0.07 percent, or from greater than 0.01 to 0.05 percent, or from greater than 0.05 to 0.15 percent, or from greater than 0.03 to 0.15 percent by weight of the solution, or from greater than 0.1 to 1 percent, or from greater than 0.1 to 0.7 percent, or from greater than 0.1 to 0.5 percent, or from greater than 0.1 to 0.2 percent, or from greater than 0.1 to 0.15 percent. Preferably, the amount of hydrogen peroxide in a formulation of the present invention throughout its shelf life is greater than 0.01 percent by weight of the total formulation.

In another aspect, the polyanionic material comprises an anionic derivative of a polysaccharide.

The present inventors surprisingly discovered that the presence of a polyanionic material in a formulation comprising hydrogen peroxide or a source thereof can provide ocular comfort to a user, which comfort is typically not experienced by the user with compositions having hydrogen peroxide or a source thereof in which such a polyanionic material is absent, especially at a relative high concentration of hydrogen peroxide.

In still another aspect, the polyanionic material included in an ophthalmic solution of the present invention is selected from the group consisting of alginic acid, carboxymethyl cellulose, carboxymethyl starch, carboxymethyl dextran, dextran sulfate, carboxymethyl chitosan, hyaluronic acid, chondroitin sulfate (e.g., chondroitin sulfate A, chondroitin sulfate B, or chondroitin sulfate C), xanthan gum, physiologically acceptable salts thereof, derivatives thereof, combinations thereof, and mixtures thereof. In one embodiment, the polyanionic material is selected from the group consisting of alginic acid, carboxymethyl cellulose, carboxymethyl starch, carboxymethyl dextran, hyaluronic acid, physiologically acceptable salts thereof, derivatives thereof, combinations thereof, and mixtures thereof. In another embodiment, the polyanionic material is selected from the group consisting of physiologically acceptable salts of alginic acid carboxymethyl cellulose, carboxymethyl starch, carboxymethyl dextran, hyaluronic acid; derivatives thereof, combinations thereof, and mixtures thereof. In still another embodiment, the polyanionic material is selected from the group consisting of physiologically acceptable salts of alginic acid, carboxymethyl starch, carboxymethyl dextran; derivatives thereof, combinations thereof, and mixtures thereof. In still another embodiment, the polyanionic material is selected from the group consisting of physiologically acceptable salts of alginic acid, carboxymethyl starch, carboxymethyl dextran, carboxymethyl chitosan, chondroitin sulfate; derivatives thereof, combinations thereof, and mixtures thereof.

In one embodiment, the amount of the polyanionic material in an ophthalmic solution of the present invention is in the range from about 0.01 to about 10 percent by weight of the solution. Alternatively, the amount of the polyanionic material is in the range from about 0.01 to about 5 percent, or from about 0.02 to about 2 percent, or from about 0.05 to about 1 percent, or from about 0.1 to about 0.5 percent by weight of the solution. In another embodiment, the polyanionic material is present in the solution in an amount sufficient to provide lubrication to an ocular surface, such as the corneal or the conjunctiva.

In another embodiment, the amount of the polyanionic material in an ophthalmic solution of the present invention is in the range from 0.01 to 10 percent by weight of the solution. Alternatively, the amount of the polyanionic material is in the range from 0.01 to 5 percent, or from 0.02 to 2 percent, or from 0.05 to 1 percent, or from 0.1 to 0.5 percent by weight of the solution.

In yet another aspect, an ophthalmic solution of the present invention is free of a material selected from the group consisting of cationic organic nitrogen-containing compounds, such as cationic organic nitrogen-containing small molecules or polymers; alcohols; and mixtures thereof.

An ophthalmic solution of the present invention can further comprise one or more other ingredients, such as therapeutic agents that target specific eye conditions, buffers, tonicity adjusting agents, surfactants, viscosity adjusting agents, chelating agents, or other components. In certain embodiments, however, an ophthalmic solution of the present invention can be desirably free of chelating agents. In some other embodiments, it may also be advantageous to exclude a chelating agent.

For example, an ophthalmic solution of the present invention can comprise a therapeutic agent such as anti-inflammatory agents, antibiotics, immunosuppressive agents, antiviral agents, antifungal agents, antiprotozoal agents, combinations thereof, or mixtures thereof. Non-limiting examples of anti-inflammatory agents include glucocorticosteroids (e.g., for short-term treatment) and non-steroidal anti-inflammatory drugs (“NSAIDs”).

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof. In one embodiment, the therapeutic agent is selected from the group consisting of difluprednate, loteprednol etabonate, prednisolone, combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycine, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyirolnitrin, siccanin, tubercidin, viridin, allylamines (e.g., butenafme, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

An ophthalmic solution of the present invention is preferably formulated in a physiologically acceptable buffer to regulate pH and tonicity in a range compatible with ophthalmic uses and with any active ingredients present therein. Non-limiting examples of physiologically acceptable buffers include phosphate buffer; a Tris-HCl buffer (comprising tris(hydroxymethyl)aminomethane and HCl); buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMP SO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

While the buffer itself is a “tonicity adjusting agent” and a “pH adjusting agent” that broadly maintains the ophthalmic solution at a particular ion concentration and pH, additional “tonicity adjusting agents” can be added to adjust or “fine tune” the final tonicity of the solution. Such tonicity adjusting agents are well known to those of skill in the art and include, but are not limited to, mannitol, sorbitol, dextrose, sucrose, urea, propylene glycol, and glycerin. Also, various salts, including halide salts of a monovalent cation (e.g., NaCl or KCl) can be utilized.

The tonicity adjusting agent, when present, is preferably in a concentration ranging from about 0.01 to about 10, or from about 0.01 to about 7, or from about 0.01 to about 5, or from about 0.1 to about 2, or from about 0.1 to about 1 percent by weight. In some embodiments where a tonicity adjusting agent is present the solution can contain a single agent or a combination of different tonicity adjusting agents. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg, or from about 250 to 300 mOsm/kg, or from about 240 to 280 mOsm/kg. For relief of dry eye symptoms, an ophthalmic formulation of the present invention may be desirably hypotonic, such as having tonicity in the range from about 200 to about 270 mOsm/kg.

Ophthalmic solutions of the present invention also can comprise one or more surfactants. Suitable surfactants can include cationic, anionic, non-ionic or amphoteric surfactants. Preferred surfactants are neutral or nonionic surfactants. Non-limiting examples of surfactants suitable for a formulation of the present invention include polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108) ), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, N.Y., 2006. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In some embodiments, the ophthalmic solutions of this invention can optionally include viscosity adjusting agents (e.g., particularly when the ophthalmic solution is intended to act as a lubricant (i.e., artificial tear)). Suitable viscosity adjusting agents for administration to an eye are well known to those of skill in the art. One or more polyanionic materials disclosed above (especially the polysaccharide-based polyanionic materials) can act as viscosity adjusting agents. However, other polysaccharides (such as the non-ionic polysaccharides) such as cellulose derivatives are commonly used to increase viscosity, and as such, can offer other advantages. Specific cellulose derivatives include, but are not limited to hydroxypropyl methyl cellulose, carboxymethyl cellulose, methyl cellulose, or hydroxyethyl cellulose. Typically, particularly when used as an artificial tear, the ophthalmic solution has a viscosity from about 1 to about 1000 centipoises (or mPa.s). As a solution, the present pharmaceutical formulation is usually dispensed in the eye in the form of an eye drop. It should be understood, however, that the present pharmaceutical formulation may also be formulated as a viscous liquid (e.g., viscosities from 50 to several thousand cps), gel, or ointment, which has even higher viscosity, for ophthalmic or non-ophthalmic uses. Furthermore, in some contact-lens related embodiments, lenses may be soaked or otherwise exposed to a pharmaceutical formulation of the present invention prior to wear.

In some embodiments, an ophthalmic formulation of the present invention can further comprise a demulcent. Polysaccharides, such as those disclosed herein above can act as demulcents. Other demulcents also can be included, such as those approved by the U.S. Food and Drug Administration (“US FDA”) and listed in 21 C.F.R. Part 349. They include hypromellose (0.2 to 2.5 percent), dextran 70 (0.1 percent when used with another polymeric demulcent listed in this regulation), gelatin (0.01 percent), liquid polyols, glycerin (0.2 to 1 percent), polyethylene glycol 300 or 400 (0.2 to 1 percent), propylene glycol (0.2 to 1 percent), polyvinyl alcohol (0.1 to 4 percent), povidone (or polyvinyl pyrrolidone, 0.1 to 2 percent). All compositions are in percent by weight of the total formulation, unless otherwise indicated.

In some other embodiments, a pharmaceutical formulation may include one or more emollients, such as those listed in 21 C.F.R. Section 349.14.

In addition to those classes of ingredients disclosed above, a pharmaceutical formulation, such as an ophthalmic solution, of the present invention can further comprise one or more other ingredients, such as antioxidants, vitamins, or other ingredients that provide added health benefits to the users. Where an ophthalmic solution is intended for contact-lens care, it can comprise other known components that are generally used for cleaning and maintenance of contact lenses, as long as these components are compatible with other ingredients in the formulation. In one embodiment, a contact-lens care solution can comprise microabrasives (e.g., polymer microbeads).

In another embodiment, a pharmaceutical formulation of the present invention can further comprise a second preservative. In some embodiments, said second preservative is polyquaternium-1. In still some embodiments, said second preservative is other than a material selected from the group consisting of cationic organic nitrogen-containing compounds and alcohols. In still some other embodiments, said second preservative is present in an amount such that the concentration of the source of hydrogen peroxide provides hydrogen peroxide at a concentration less than about 0.1%, or less than about 0.03%, or less than about 0.01% by weight of the total formulation. In still some other embodiments, said second preservative is polyquaternium-1 and is present in an amount such that the concentration of the source of hydrogen peroxide provides hydrogen peroxide at a concentration less than about 0.1%, or less than about 0.03%, or less than about 0.01% by weight of the total formulation. In still another embodiment, said second preservative comprises another oxidative preservative, such as stabilized oxychloro complex (an equilibrium mixture of oxychloro species). In still another embodiment, such a stabilized oxychloro complex is present in an amount from about 0.001 to about 0.01% by weight of the total formulation.

In another aspect, the present invention provides a method for preparing a pharmaceutical formulation that comprises at least a polyanionic material and at least a source of hydrogen peroxide. The method comprises adding said at least a polyanionic material and at least a source of hydrogen peroxide to a formulation.

### Formulation Compounding Procedure

In one aspect, a pharmaceutical formulation of the present invention can be prepared by a method comprising the step of: (a) adding a source of hydrogen peroxide into a vessel containing 80-90 percent of a desired volume of purified water; (b) adding other desired ingredients, such as therapeutic, nutritional, or prophylactic ingredients, which target a desired physiological condition, to form a first mixture; (c) adding at least a polyanionic material to the first mixture to form a second mixture; (d) adding purified water to the vessel to bring the total volume of the second mixture to 100 percent of the desired volume; and (e) mixing the contents of the vessels to produce the pharmaceutical formulation. The method can further comprise subjecting the pharmaceutical formulation to sterilization by heating, autoclaving and/or filtration through a desired filter. Optionally, the method also can comprise adding one or more additional ingredients to the second mixture, which additional ingredients are selected from the group consisting of buffers, tonicity adjusting agents, surfactants, chelating agents, demulcents, emollients, antioxidants, viscosity adjusting agents, vitamins, other ingredients that provide added health benefits to the users, and mixtures thereof. Procedure for evaluating the preservative efficacy (“PE”) of a pharmaceutical formulation of the present invention against microorganisms

The microorganisms against which the PE of a pharmaceutical formulation of the present invention is evaluated are S. aureus, E. coli, P. aeruginosa, C. albicans, and A. niger. This procedure applies to the US FDA premarket notification (510(k)) guidance document and ISO/DIS 14730 standard preservative efficacy testing with a 14-day rechallenge. The evaluations were conducted with 3 separate lots of each test solution for each microorganism. Each lot was tested with a different preparation of each microorganism.

Bacterial cells were grown on Tryptic Soy Agar (“TSA”) slants at a temperature in the range from 30 to 35° C. in an incubator for a time period from 18 to 24 hours. Fungal cells were grown on Sabouraud Dextrose Agar (“SDA”) slants at a temperature in the range from 20° C. to 25° C. in an incubator for a time period of 2 to 7 days. Cells were harvested in saline solution (5-10 ml, USP, 0.9% saline, with or without 0.1% Tween 80 surfactant, which was added to each agar slant, followed by gentle agitation with a sterile cotton swab. The cell suspensions were aseptically dispensed into separate sterile polypropylene centrifuge tubes. Cells were harvested by centrifugation at 3000 rpm for 10 minutes, washed one time, and suspended in Saline TS to a concentration of 2×10⁸ cells per ml.

The cell suspension (0.1 ml) was diluted with 20 ml of the test solution to reach a final concentration of from 1.0×10⁵ to 1.0×10⁶ colony-forming units (“CFU”). Phosphate Buffered Saline (“PBS”) was used as a control solution. The inoculated test and control solutions were incubated at a temperature ranging from 20° C. to 25° C. in static culture. At time zero, 1 ml of PBS (USP, pH 7.2) from the control solution was diluted with 9 ml of PBS and serially diluted cells were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

Similarly, at days 7 and 14, a one-milliliter volume from a test solution was added into 9 ml of Dey-Engley neutralizing broth (“DEB”) and serially diluted in DEB and plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20° C. to 25° C. for a period ranging from 2 to 7 days. Developing colonies were counted.

Immediately following the day 14 sampling, test solutions were re-inoculated to give final concentrations of from 1.0×10⁴ to 1.0×10⁵ of each microorganism. At time zero, 1 ml from the inoculum control was added to 9 ml of PBS and subsequent serial dilutions were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

At days 21 and 28, 1 ml from the test articles was added to 9 ml of DEB and again, serial dilutions were plated in triplicate on TSA. Plates were incubated at a temperature ranging from 30 to 35° C. for a period ranging from 2 days to 4 days and developing colonies counted.

Based on the acceptance criteria for bacteria, a solution is acceptable if the concentration of viable bacteria, recovered per milliliter, is reduced by at least 3 logs at day 14, and after a rechallenge at day 14, the concentration of bacteria is reduced by at least 3 logs by day 28. In addition, the solution is acceptable if the concentration of viable yeasts and molds, recovered per milliliter of the solution, remains at or below the initial concentration (within an experimental uncertainty of ±0.5 log) at day 14, and after a rechallenge at day 14, the concentration of viable yeasts and molds remains at or below the initial concentration (within an experimental uncertainty of ±0.5 log) at day 28.

The results at the fourteenth and twenty-eighth days for the tested solutions are shown in the following tables as log reduction in the concentration of the applicable microorganism.

### EXAMPLE 1

### First Formulation

The first formulation had the following composition.

| | % w/w (except pH |
|---|---|
| Ingredient | and Osmolality) |
| Sodium Borate | 0.06 |
| Boric Acid | 0.7 |
| Glycerin | 1 |
| Sodium Alginate | 0.25 |
| Urea Hydrogen Peroxide | 0.1 |
| EDTA | 0.05 |
| pH | 6.66 |
| Osmolality | 262 mOsm/kg |
| PE | Passed |

### PE Test Result

| | | First Challenge on Day | First Challenge |
|---|---|---|---|
| | Days after | of Formulation | after 12 |
| Organism | Challenge | Preparation | Months in Storage |
| S. aureus | 14 days | >4.9 | >4.8 |
| | 28 days | >3.8 | >4.8 |
| P. aeruginosa | 14 days | >4.7 | >4.7 |
| | 28 days | >3.7 | 4.5 |
| E. coli | 14 days | >4.8 | >4.7 |
| | 28 days | >3.8 | >4.8 |
| C. albicans | 14 days | >4.9 | >4.8 |
| | 28 days | >3.8 | >4.8 |
| A. niger | 14 days | 1.5 | 1.4 |
| | 28 days | 1.3 | 1.4 |

### EXAMPLE 2

### Second Formulation

The second formulation had the following composition.

| | % w/w (except pH |
|---|---|
| Ingredient | and Osmolality) |
| Sodium Borate | 0.06 |
| Boric Acid | 0.7 |
| Glycerin | 0.6 |
| Propylene Glycol | 0.6 |
| Sodium Alginate | 0.25 |
| Urea Hydrogen Peroxide | 0.1 |
| EDTA | 0.05 |
| pH | 6.75 |
| Osmolality | 300 mOsm/kg |
| PE | Passed |

### PE Test Result

| | | First | | First |
|---|---|---|---|---|
| | | Challenge | | Challenge |
| | | on Day of | First Challenge | after 26 |
| | Days after | Formulation | after 12 Months | Months in |
| Organism | Challenge | Preparation | in Storage | Storage |
| S. aureus | 14 days | >4.9 | >4.8 | >4.7 |
| | 28 days | >3.8 | >4.8 | >4.7 |
| P. aeruginosa | 14 days | >4.7 | >4.7 | >4.6 |
| | 28 days | >3.7 | >4.7 | >4.6 |
| E. coli | 14 days | 4.8 | >4.7 | 4.3 |
| | 28 days | >3.8 | >4.7 | >4.6 |
| C. albicans | 14 days | >4.9 | >4.8 | >4.8 |
| | 28 days | >3.8 | >4.8 | >4.8 |
| A. niger | 14 days | 1.4 | 1.2 | 1.9 |
| | 28 days | 0.8 | 1.5 | 2.2 |

### EXAMPLE 3

### Third Formulation

The third formulation had the following composition.

| | % w/w (except pH |
|---|---|
| Ingredient | and Osmolality) |
| Sodium Borate | 0.06 |
| Boric Acid | 0.7 |
| Glycerin | 1 |
| Sodium Alginate | 0.25 |
| Urea Hydrogen Peroxide | 0.25 |
| pH | 6.74 |
| Osmolality | 291 mOsm/kg |
| PE | Passed |

### PE Test Result

| | | First Challenge on Day | First Challenge |
|---|---|---|---|
| | Days after | of Formulation | after 12 |
| Organism | Challenge | Preparation | Months in Storage |
| S. aureus | 14 days | >4.9 | >4.8 |
| | 28 days | >3.8 | 4.8 |
| P. aeruginosa | 14 days | >4.7 | >4.7 |
| | 28 days | >3.7 | >4.7 |
| E. coli | 14 days | >4.8 | >4.7 |
| | 28 days | >3.8 | >4.7 |
| C. albicans | 14 days | >4.9 | >4.8 |
| | 28 days | >3.8 | >4.8 |
| A. niger | 14 days | >4.6 | >4.5 |
| | 28 days | >3.6 | >4.5 |

### EXAMPLE 4

### Fourth Formulation

The fourth formulation had the following composition.

| | % w/w (except pH |
|---|---|
| Ingredient | and Osmolality) |
| Sodium Borate | 0.06 |
| Boric Acid | 0.7 |
| Glycerin | 1 |
| Sodium Alginate | 0.25 |
| Urea Hydrogen Peroxide | 0.25 |
| EDTA | 0.05 |
| pH | 6.56 |
| Osmolality | 295 mOsm/kg |
| PE | Passed |

### PE Test Result

| | | | | First |
|---|---|---|---|---|
| | | First Challenge | First Challenge | Challenge |
| | | on Day of | after 12 | after 26 |
| | Days after | Formulation | Months | Months |
| Organism | Challenge | Preparation | in Storage | in Storage |
| S. aureus | 14 days | >4.9 | >4.8 | >4.7 |
| | 28 days | >3.8 | >4.8 | >4.7 |
| P. aeruginosa | 14 days | >4.7 | >4.7 | >4.6 |
| | 28 days | >3.7 | >4.7 | >4.6 |
| E. coli | 14 days | >4.8 | >4.7 | >4.8 |
| | 28 days | >3.8 | >4.7 | >4.8 |
| C. albicans | 14 days | >4.9 | >4.8 | >4.5 |
| | 28 days | >3.8 | >4.8 | >4.5 |
| A. niger | 14 days | >4.6 | 3.8 | |
| | 28 days | >3.6 | >4.5 | |

### EXAMPLE 5

### Fifth Formulation

The fifth formulation had the following composition.

| | % w/w (except pH |
|---|---|
| Ingredient | and Osmolality) |
| Sodium Borate | 0.06 |
| Boric Acid | 0.7 |
| Glycerin | 1 |
| Sodium Alginate | 0.25 |
| Urea Hydrogen Peroxide | 0.1 |
| EDTA | 0.1 |
| pH | 6.49 |
| Osmolality | 267 |
| PE | Passed on day of |
| | preparation, but failed after |
| | 12 months in storage |

### PE Test Result

| | | | | First |
|---|---|---|---|---|
| | | First Challenge | First Challenge | Challenge |
| | | on Day of | after | after |
| | Days after | Formulation | 12 Months | 26 Months |
| Organism | Challenge | Preparation | in Storage | in Storage |
| S. aureus | 14 days | >4.9 | 1.7 | 1.0 |
| | 28 days | >3.8 | >4.8 | >4.7 |
| P. aeruginosa | 14 days | >4.7 | >4.7 | >4.6 |
| | 28 days | >3.7 | >4.7 | >4.6 |
| E. coli | 14 days | >4.8 | 1.1 | 0.7 |
| | 28 days | >3.8 | >4.7 | 1.6 |
| C. albicans | 14 days | >4.9 | 0.4 | 0.4 |
| | 28 days | >3.8 | 3.8 | 2.8 |
| A. niger | 14 days | 1.3 | 1.2 | 1.9 |
| | 28 days | 0.7 | 1.2 | 2.2 |

### EXAMPLE 6

### Sixth Formulation

The sixth formulation had the following composition.

| | % w/w/ (except pH |
|---|---|
| Ingredient | and Osmolality) |
| Sodium Borate | 0.014 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.2 |
| pH | 6.49 |
| Osmolality | 277 |
| PE | Passed |

### PE Test Result

| | | First Challenge on Day | First Challenge |
|---|---|---|---|
| | Days after | of Formulation | after 4 |
| Organism | Challenge | Preparation | Months in Storage |
| S. aureus | 14 days | >4.8 | No data |
| | 28 days | >4.8 | No data |
| P. aeruginosa | 14 days | >4.6 | No data |
| | 28 days | >4.6 | No data |
| E. coli | 14 days | >4.7 | No data |
| | 28 days | >4.7 | No data |
| C. albicans | 14 days | >4.9 | No data |
| | 28 days | >4.9 | No data |
| A. niger | 14 days | >4.5 | No data |
| | 28 days | >4.5 | No data |

### EXAMPLE 7

### Seventh Formulation

The seventh formulation had the following composition.

| | % w/w/ (except pH |
|---|---|
| Ingredient | and Osmolality) |
| Sodium Borate | 0.014 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.15 |
| pH | 6.52 |
| Osmolality | 270 |
| PE | Passed |

### PE Test Result

| | | First Challenge on Day | First Challenge |
|---|---|---|---|
| | Days after | of Formulation | after 4 |
| Organism | Challenge | Preparation | Months in Storage |
| S. aureus | 14 days | >4.8 | >4.7 |
| | 28 days | >4.8 | >4.7 |
| P. aeruginosa | 14 days | >4.6 | >4.6 |
| | 28 days | >4.6 | >4.6 |
| E. coli | 14 days | >4.7 | >4.7 |
| | 28 days | >4.7 | >4.7 |
| C. albicans | 14 days | >4.9 | >4.8 |
| | 28 days | >4.9 | >4.8 |
| A. niger | 14 days | 4.5 | >4.4 |
| | 28 days | >4.5 | >4.4 |

### EXAMPLE 8

### Eighth Formulation

The eighth formulation had the following composition.

| | % w/w/ |
|---|---|
| Ingredient | (except pH and Osmolality) |
| Sodium Borate | 0.014 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.1 |
| pH | 6.55 |
| Osmolality | 257 |
| PE | Passed |

### PE Test Result

| | | First Challenge on Day | First |
|---|---|---|---|
| | Days after | of Formulation | Challenge after 4 |
| Organism | Challenge | Preparation | Months in Storage |
| S. aureus | 14 days | >4.8 | >4.7 |
| | 28 days | >4.8 | >4.7 |
| P. aeruginosa | 14 days | >4.6 | >4.6 |
| | 28 days | >4.6 | >4.6 |
| E. coli | 14 days | >4.7 | >4.7 |
| | 28 days | >4.7 | >4.7 |
| C. albicans | 14 days | >4.9 | >4.8 |
| | 28 days | >4.9 | >4.8 |
| A. niger | 14 days | 2.3 | 2.1 |
| | 28 days | >4.5 | 4.1 |

### EXAMPLE 9

### Ninth Formulation

The ninth formulation had the following composition.

| | % w/w/ |
|---|---|
| Ingredient | (except pH and Osmolality) |
| Sodium Borate | 0.014 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.05 |
| pH | 6.54 |
| Osmolality | 246 |
| PE | Passed |

### PE Test Result

| | | First Challenge on Day | First |
|---|---|---|---|
| | Days after | of Formulation | Challenge after 4 |
| Organism | Challenge | Preparation | Months in Storage |
| S. aureus | 14 days | >4.8 | >4.7 |
| | 28 days | >4.8 | >4.7 |
| P. aeruginosa | 14 days | >4.6 | >4.6 |
| | 28 days | >4.6 | >4.6 |
| E. coli | 14 days | 4.7 | >4.7 |
| | 28 days | >4.7 | >4.7 |
| C. albicans | 14 days | >4.9 | >4.8 |
| | 28 days | >4.9 | >4.8 |
| A. niger | 14 days | 1.3 | 1.9 |
| | 28 days | 3.6 | 2.1 |

### EXAMPLE 10

### Tenth Formulation

The tenth formulation had the following composition.

| | % w/w/ |
|---|---|
| Ingredient | (except pH and Osmolality) |
| Sodium Borate | 0.014 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.03 |
| pH | 6.57 |
| Osmolality | 243 |
| PE | Passed |

### PE Test Result

| | | First Challenge on Day | First |
|---|---|---|---|
| | Days after | of Formulation | Challenge after 4 |
| Organism | Challenge | Preparation | Months in Storage |
| S. aureus | 14 days | >4.8 | >4.7 |
| | 28 days | >4.8 | >4.7 |
| P. aeruginosa | 14 days | >4.6 | >4.6 |
| | 28 days | >4.6 | >4.6 |
| E. coli | 14 days | >4.7 | >4.7 |
| | 28 days | >4.7 | >4.7 |
| C. albicans | 14 days | >4.9 | >4.8 |
| | 28 days | >4.9 | >4.8 |
| A. niger | 14 days | 1.2 | 2.2 |
| | 28 days | 3.2 | 2.9 |

In some embodiments, one or more pharmaceutical active ingredients suitable for ophthalmic administration are included in a pharmaceutical formulation of the present invention for treatment or control of an ophthalmic disorder or disease. Non-limiting examples of such formulations are shown below.

### EXAMPLE 11

### Ophthalmic Formulation with Anti-Inflammatory Drug

The following ingredients are combined to produce such a formulation.

| Ingredient | % w/w |
|---|---|
| Sodium Borate | 0.02 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.1 |
| Diclofenac Sodium | 0.5 |

### EXAMPLE 12

### Ophthalmic Formulation for Treating or Controlling High Intraocular Pressure

The following ingredients are combined to produce an exemplary formulation for treating or controlling high intraocular pressure.

| Ingredient | % w/w |
|---|---|
| Sodium Borate | 0.05 |
| Boric Acid | 0.6 |
| Glycerin | 0.75 |
| Propylene Glycol | 0.3 |
| Sodium Alginate (Protanal LF200M) | 0.3 |
| Urea Hydrogen Peroxide | 0.07 |
| Timolol Maleate | 0.5 |
| Brimonidine Tartrate | 0.25 |

### EXAMPLE 13

### Ophthalmic Formulation for Treating or Controlling Eye Infection

The following ingredients are combined to produce such a formulation.

| Ingredient | % w/w |
|---|---|
| Sodium Borate | 0.02 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.1 |
| Moxifloxacin | 0.5 |

### EXAMPLE 14

### Ophthalmic Formulation for Treating or Controlling Eye Infection

The following ingredients are combined to produce such a formulation.

| Ingredient | % w/w |
|---|---|
| Sodium Borate | 0.02 |
| Boric Acid | 0.5 |
| Glycerin | 1 |
| Propylene Glycol | 0.2 |
| Sodium Alginate (Protanal LF200M) | 0.25 |
| Urea Hydrogen Peroxide | 0.1 |
| Gatifloxacin | 0.5 |

### EXAMPLE 15

### Ophthalmic Formulation for Treating or Controlling Eye Infection

The following ingredients are combined to produce such a formulation.

| Ingredient | % w/w |
|---|---|
| Sodium Borate | 0.03 |
| Boric Acid | 0.35 |
| Glycerin | 0.6 |
| Propylene Glycol | 0.6 |
| Sodium Alginate (Protanal LF200M) | 0.25 |
| EDTA | 0.05 |
| Urea Hydrogen Peroxide | 0.1 |
| Ciprofloxacin | 0.15 |
| 7-[(3R)-3-aminohexahydro-1H-azepin- | 0.2 |
| 1-yl]-8-chloro-1-cyclopropyl-6-fluoro- | |
| 1,4-dihydro-4-oxo-3- | |
| quinolinecarboxylic acid | |
| monohydrochloride | |

### EXAMPLE 16

### Ophthalmic Formulation for Treating or Controlling Eye Allergy

The following ingredients are combined to produce an exemplary formulation for treating or controlling eye allergy.

| Ingredient | % w/w |
|---|---|
| Sodium Borate | 0.06 |
| Boric Acid | 0.7 |
| Glycerin | 0.6 |
| Propylene Glycol | 0.6 |
| Sodium Alginate (Protanal LF200M) | 0.25 |
| Urea Hydrogen Peroxide | 0.1 |
| Ketotifen Fumarate | 0.025 |

### EXAMPLE 17

### Ophthalmic Formulation for Treating or Controlling Eye Allergy

The following ingredients are combined to produce an exemplary formulation for treating or controlling eye allergy.

| Ingredient | % w/w |
|---|---|
| Sodium Borate | 0.06 |
| Boric Acid | 0.7 |
| Glycerin | 0.6 |
| Propylene Glycol | 0.6 |
| Sodium Alginate (Protanal LF200M) | 0.25 |
| Urea Hydrogen Peroxide | 0.07 |
| Olopatadine Hydrochloride | 0.1 |

### EXAMPLE 18

### Ophthalmic Formulation for Treating or Controlling Eye Infection

The following ingredients are combined to produce an exemplary formulation for treating or controlling eye infection.

| Ingredient | % w/w |
|---|---|
| Sodium Borate | 0.06 |
| Boric Acid | 0.7 |
| Glycerin | 0.6 |
| Propylene Glycol | 0.6 |
| Sodium Alginate (Protanal LF200M) | 0.25 |
| Urea Hydrogen Peroxide | 0.1 |
| Acyclovir | 0.05 |

### EXAMPLE 19

### Ophthalmic Formulation for Treating or Controlling Eye Infection

The following ingredients are combined to produce an exemplary formulation for treating or controlling eye infection. The polyanionic material included in this formulation is Carbomer 940. Carbomers are also known as carbopol or carboxypolymethylene. Carbomers are synthetic high molecular weight polymers of acrylic acid cross-linked with either allylsucrose or allylethers of pentaerythritol. They contain between 56 and 68% carboxylic groups.

| Ingredient | % w/w |
|---|---|
| Sodium Borate | 0.06 |
| Boric Acid | 0.7 |
| Glycerin | 0.6 |
| Propylene Glycol | 0.6 |
| Carbomer 940 | 0.25 |
| Urea Hydrogen Peroxide | 0.1 |
| Acyclovir | 0.05 |

### EXAMPLE 20

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Glycerin | 0.6 |
| Propylene Glycol | 0.6 |
| Carbomer 940 | 0.25 |
| Urea Hydrogen Peroxide | 0.1 |
| Loteprednol Etabonate | 0.5 |

### EXAMPLE 21

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Glycerin | 0.6 |
| Propylene Glycol | 0.6 |
| Carbomer 940 | 0.25 |
| Urea Hydrogen Peroxide | 0.1 |
| Loteprednol Etabonate | 0.5 |
| Tobramycin | 0.3 |

### EXAMPLE 21

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Glycerin | 0.6 |
| Propylene Glycol | 0.6 |
| Carbopol 980 | 0.25 |
| Urea Hydrogen Peroxide | 0.1 |
| Dexamethasone | 0.1 |

### EXAMPLE 22

### Ophthalmic Formulation for Treating or Controlling Intraocular Pressure

The following ingredients are combined to produce an exemplary formulation for treating or controlling intraocular pressure.

| Ingredient | % w/w |
|---|---|
| Sodium Borate | 0.06 |
| Boric Acid | 0.7 |
| Glycerin | 0.6 |
| Propylene Glycol | 0.6 |
| Sodium Alginate | 0.25 |
| Urea Hydrogen Peroxide | 0.1 |
| Dorzolamide Hydrochloride | 2 |
| Timolol Maleate | 0.5 |

### EXAMPLE 23

### Formulation Comprising a Second Preservative

The following ingredients are combined to produce an exemplary formulation.

| Ingredient | % w/w |
|---|---|
| Sodium Borate | 0.06 |
| Boric Acid | 0.7 |
| Glycerin | 0.6 |
| Propylene Glycol | 0.6 |
| Sodium Alginate | 0.25 |
| Urea Hydrogen Peroxide | 0.03 |
| Polyquaternium-1 | 0.05 |

### EXAMPLE 24

### Formulation Comprising a Second Preservative

The following ingredients are combined to produce an exemplary formulation.

| Ingredient | % w/w |
|---|---|
| Sodium Borate | 0.06 |
| Boric Acid | 0.7 |
| Propylene Glycol | 1 |
| Sodium Alginate | 0.4 |
| Urea Hydrogen Peroxide | 0.05 |
| Stabilized Oxychloro Complex | 0.01 |

### Cytotoxicty Testing

Cytotoxicity assay was carried out for selected formulations shown in the forgoing section. The in vitro fluorescein permeability cytotoxicity assay involved exposing a monolayer of either Madin-Darby canine kidney (“MDCK”) cells or human corneal epithelial cells (“HCEC”) to various test solutions for 30 minutes. The monolayer of cells that were exposed to the test solutions are then treated with sodium fluorescein. The sodium fluorescein that penetrated the tight junctions of the cells was detected using fluorescence spectroscopy. The fluorescence analysis was also done after 24 hours of incubation to evaluate the recovery of the epithelial monolayer. The results shown in Table I (below) indicate that the tested formulations containing urea hydrogen peroxide as the preservative system exhibited no or very low cytotoxicity profiles for both cell lines. In addition, the cytotoxicity profiles are lower than those of Refresh Tears® and Systane free®, which are both commercial products claiming to contain gentle preservative systems.

TABLE I
Fluorescence Units measured at 485 nm excitation and 535 nm emission
after a 30-minute exposure to solution and 24 hours of recovery

| 30 minute exposure | | Standard | 24 hr. | 24 hr. |
|---|---|---|---|---|
| Solution | Average | Dev. | Recovery | St. Dev. |
| | MDCK | | | |
| HBSS⁽¹⁾ | 72.40 | 46.14 | 38.33 | 9.71 |
| 0.05% SDS/HBSS | 1449.67 | 157.64 | 860.17 | 838.69 |
| OptiFree Express ® | 166.50 | 125.37 | 60.33 | 6.09 |
| Formulation of Example 6 | 38.40 | 9.58 | 59.60 | 17.67 |
| Formulation of Example 7 | 34.83 | 2.99 | 58.00 | 11.66 |
| Formulation of Example 8 | 39.50 | 5.50 | 48.83 | 9.33 |
| Formulation of Example 9 | 35.50 | 2.88 | 62.33 | 10.91 |
| Formulation of Example 10 | 52.67 | 36.00 | 57.50 | 11.95 |
| Systane Free ® | 45.40 | 9.13 | 74.50 | 23.51 |
| Refresh Tears ® | 34.00 | 3.03 | 73.67 | 26.34 |
| Physiological Saline | 42.67 | 9.79 | 67.83 | 22.25 |
| | HCEC | | | |
| HBSS⁽¹⁾ | 822.83 | 94.23 | 156.00 | 23.61 |
| 0.05% SDS/HBSS | 7018.17 | 843.16 | 8475.33 | 620.14 |
| OptiFree Express ® | 1403.50 | 93.89 | 935.67 | 246.68 |
| Formulation of Example 6 | 299.33 | 36.58 | 709.83 | 172.80 |
| Formulation of Example 7 | 373.00 | 80.55 | 663.17 | 243.17 |
| Formulation of Example 8 | 322.67 | 58.22 | 651.33 | 149.11 |
| Formulation of Example 9 | 222.67 | 10.54 | 301.17 | 47.06 |
| Formulation of Example 10 | 245.67 | 48.24 | 207.17 | 37.40 |
| Systane Free ® | 424.00 | 95.07 | 146.67 | 10.25 |
| Refresh Tears ® | 355.50 | 129.40 | 145.00 | 15.99 |
| Physiological Saline | 374.50 | 40.59 | 113.83 | 8.35 |

Note:
⁽¹⁾Hank's Balanced Salt Solution

### Ocular Irritation Testing in Rabbit Model

This study is designed to evaluate the alginate based formulation containing 0.18% urea hydrogen peroxide. Hydrogen peroxide is present in this formulation at a concentration of about 600 ppm. Six healthy adult rabbits with clinically normal eyes were used for the course of the study. The eyes were examined with fluorescein stain under a slit lamp prior to commencement of the study on Day 0 and following the final (6th) instillation of the formulation on Day 5. The eyes were also examined macroscopically daily and scored using the Draize method. Observations were also made daily for signs of systemic toxicity and for any outward signs of pain or discomfort. The results of all testing are summarized in Table II.

TABLE II

| | Observation (Draize score⁽¹⁾) | Observation (Draize score⁽¹⁾) |
|---|---|---|
| Test | Test eye (Right) | Untreated Control Eye (Left) |
| Draize (Cornea/ | 0/0/0 for all test animals | 0/0/0 for all test animals |
| Iris/Conjunctivae) Pre-Tx | | |
| Draize (Cornea/ | 0/0/0 for all test animals | 0/0/0 for all test animals |
| Iris/Conjunctivae) Day 1 | | |
| Draize (Cornea/ | 0/0/0 for all test animals | 0/0/0 for all test animals |
| Iris/Conjunctivae) Day 2 | | |
| Draize (Cornea/ | 0/0/0 for all test animals | 0/0/0 for all test animals |
| Iris/Conjunctivae) Day 3 | | |
| Draize (Cornea/ | 0/0/0 for all test animals | 0/0/0 for all test animals |
| Iris/Conjunctivae) Day 4 | | |
| Draize (Cornea/ | 0/0/0 for all test animals | 0/0/0 for all test animals |
| Iris/Conjunctivae) Day 5 | | |

Note:
⁽¹⁾A Draize score of 0 indicates the following:
Cornea = no observed opacity
Iris = lack of swelling, congestion, circumcorneal injection, or sluggish reaction to light
Conjunctivae = Vessels normal, no observed swelling, and no discharge. For an in-depth description of the test, see J. H. Draize et al., “Method for the Study of Irritation and Toxicity of Substances Applied Topically to the Skin and Mucous Membranes,” J. Pharmacol. Exp. Ther., Vol. 82, 377 (1944).

There were no abnormalities reported during the slit lamp observations conducted prior to the first instillation or following the last instillation in any of the test or control eyes. There was no observable weight loss in any of the animals from Day 0 to Day 5. Finally, no rabbit exhibited observable systemic abnormalities or behaviors indicative of discomfort post-test dosing or at any other time.

Comparison between a Formulation of the Present Invention and a Commercial Eye Drop

A study was performed to evaluate the safety and tolerability of a formulation of the present invention (BL-700-DDE22) in volunteers compared to Systane Lubricant Eye Drops, a commercial eye drop for relief of dry eye, preserved with polyquaternium-1.

| | BL-700-DDE22 |
|---|---|
| Ingredient | (% w/w) |
| Boric Acid | 0.5000 |
| Sodium Borate | 0.01400 |
| Glycerin | 0.6000 |
| Propylene Glycol | 0.6000 |
| Protanal LF 200M | 0.2500 |
| (Alginate, medium | |
| viscosity) | |
| Urea Hydrogen | 0.162 |
| Peroxide | (~575 ppm H₂O₂) |
| Purified Water | q.s. to 100% |

Thirty-six (36) subjects completed a four-hour, dispensing study comparing formulation BL-700-DDE22 to Systane Lubricant Eye Drops (the control drop). Compared to the control drop, BL-700-DDE22 exhibited statistically significant better visual quality. There were no statistically significant differences noted between BL-700-DDE22 and the control drop with respect to comfort, visual quality, foreign body sensation, itchiness, scratchiness/grittiness, forced-choice preference, and normalized corneal and conjunctival staining severity and extent. One subject was discontinued because of missing drop instillation between visits. No adverse events occurred during the course of the study.

### Procedure

Prior to drop instillation, corneal and conjunctival staining and habitual Snellen visual acuity was assessed. Each subject had a randomly assigned test/control drop instilled contralaterally. Analog sting/burn, foreign body sensation, scratchiness/grittiness, itchiness, visual quality and comfort were assessed by the subject. Each subject was instructed to instill the test/control drops every hour until their final visit. On the final visit, a final test/control drop was instilled (a total of 5 drop instillations over 4 hours). The previous assessments were repeated and a forced-choice preference (with reason for choice) was conducted. Corneal and conjunctival staining and habitual Snellen visual acuity were reassessed. Testing took place during a period of 6 days.

### Statistical Analysis

The two-way repeated measures ANOVA was used to test for differences in each of the parametric dependent variables. Non-parametric data were analyzed using the Wilcoxon Matched Pairs test. Forced-choice data was assessed using the chi-square test. Differences at the α≦0.05 level were considered statistically significant.

There were no statistically significant differences for mean comfort with respect to time, drop, or the time/drop interaction. Of the thirty-six subjects who completed the study and were asked to make a choice, sixteen subjects preferred BL-700-DDE22, thirteen subjects preferred the control drop and seven subjects had no preference. Analysis was conducted on the twenty-nine subjects who had a preference. The difference was not statistically significant (χ² test, p>0.57).

In another aspect, an ophthalmic solution of the present invention comprising a polyanionic material, a source of hydrogen peroxide, boric acid, and at least a suitable ophthalmic active ingredient can be used to treat ocular conditions such as dry eye, inflammation, allergy, or infection of the eye.

In still another aspect, the present invention provides methods of making and using a pharmaceutical formulation of the present invention. Any of the materials, compounds, and ingredients disclosed herein is applicable for use with or inclusion in any method of the present invention.

In still another aspect, the present invention provides a method for making a pharmaceutical formulation. The method comprises providing at least a polyanionic material and at least a source of hydrogen peroxide in the pharmaceutical formulation. In one embodiment, the method comprises: (a) providing an initial formulation; and (b) adding said at least a polyanionic material and said at least a source of hydrogen peroxide to the initial formulation to produce the pharmaceutical formulation. In another embodiment, the method further comprises adding another ingredient selected from the group consisting of therapeutic agents, buffers, tonicity adjusting agents, surfactants, viscosity adjusting agents, and other agents to the pharmaceutical formulation. The therapeutic agents can be selected from the group of anti-inflammatory agents, antibiotics, immunosuppressive agents, anti-allergic agents, antiviral agents, antifungal agents, and antiprotozoal agents. In still another embodiment, the source of hydrogen peroxide comprises a compound that is soluble in an aqueous medium. Non-limiting examples of each of these classes of agents, compounds, and ingredients are disclosed throughout the present specification.

In still another aspect, the present invention provides a method for making a pharmaceutical formulation. The method comprises providing at least a polyanionic material, boric acid, and at least a source of hydrogen peroxide in the pharmaceutical formulation. In one embodiment, the method comprises: (a) providing an initial formulation comprising boric acid and said at least a source of hydrogen peroxide; and (b) adding said at least a polyanionic material to the initial formulation to produce the pharmaceutical formulation. The method can further comprise adding an ophthalmically active agent to the pharmaceutical formulation.

In still another aspect, the present invention provides a method for providing safety, or comfort, or both to users of a pharmaceutical formulation. The method comprises adding at least a polyanionic material and at least a source of hydrogen peroxide to the pharmaceutical formulation. In one embodiment, the source of hydrogen peroxide is a compound that generates hydrogen peroxide in an aqueous medium. In another embodiment, the polyanionic material is selected from the group consisting of alginic acid, carboxymethyl cellulose, carboxymethyl starch, carboxymethyl dextran, dextran sulfate, carboxymethyl chitosan, hyaluronic acid, chondroitin sulfate (e.g., chondroitin sulfate A, chondroitin sulfate B, or chondroitin sulfate C), xanthan gum, physiologically acceptable salts thereof, derivatives thereof, combinations thereof, and mixtures thereof. In another embodiment, the polyanionic material is selected from the group consisting of alginic acid, carboxymethyl cellulose, carboxymethyl starch, carboxymethyl dextran, hyaluronic acid, physiologically acceptable salts thereof, derivatives thereof, combinations thereof, and mixtures thereof. In still another embodiment, the polyanionic material is selected from the group consisting of physiologically acceptable salts of alginic acid, carboxymethyl cellulose, carboxymethyl starch, carboxymethyl dextran, hyaluronic acid; derivatives thereof; combinations thereof; and mixtures thereof.

In yet another aspect, the present invention provides a method for treating, controlling, or preventing a condition of an eye that manifests dryness (for lack of adequate tear production), allergy, irritation, or inflammation. The method comprises topically administering to the eye an effective amount of an ophthalmic solution that comprises at least a polyanionic material and at least a source of hydrogen peroxide to relieve such dryness, allergy, irritation, or inflammation. In one embodiment, the method is used for treating a dry eye condition. In another embodiment, the method for treating or relieving symptoms of dry eye comprises administering to an ocular surface an effective amount of an ophthalmic solution that comprises a polyanionic material, a source of hydrogen peroxide, a demulcent, a tonicity adjusting agent, and a buffering agent. The concentration of each of polyanionic material and source of hydrogen peroxide is selected from among the ranges disclosed herein.

In a further aspect, the present invention provides a method for treating an ophthalmic device. The method comprises contacting the ophthalmic device with an ophthalmic solution comprising at least a polyanionic material and at least a source of hydrogen peroxide. In one embodiment, the ophthalmic solution has the capability to clean, disinfect, and wet or rewet the ophthalmic device. In another embodiment, the ophthalmic solution further comprises an amount of boric acid. The ophthalmic solution comprises a polyanionic material, a source of hydrogen peroxide, a surfactant, and a tonicity adjusting agent. The ophthalmic solution can further comprise a buffering agent.

In still a further aspect, the ophthalmic device is a contact lens.

In a further aspect, the present invention provides a use of at least a polyanionic material and at least a source of hydrogen peroxide for the preparation of a pharmaceutical formulation, such as an ophthalmic solution. In some embodiments of the present invention, the preparation can further include the use of additional ingredients, such as therapeutic agents, buffers, tonicity adjusting agents, surfactants, viscosity adjusting agents, antioxidants, other agents, combinations thereof, or mixtures thereof.

In yet another aspect, the source of hydrogen peroxide is included in a formulation in an amount sufficient to reduce the concentration of bacteria by at least 3 logs reduction at the fourteenth day after challenge with said bacteria, and to reduce the concentration of bacteria by at least 3 logs reduction at the twenty-eighth day after rechallenge with said bacteria at the fourteenth day. In addition, in further embodiments, the amount of the source of hydrogen peroxide is also sufficient to keep the concentration of yeasts and molds at the fourteenth day after challenge with said yeasts and molds at or below the initial concentration, and to keep the concentration of yeasts and molds at the twenty-eighth day after rechallenge with said yeasts and molds at the fourteenth day at or below the initial concentration.

In a further aspect, the source of hydrogen peroxide is included in a formulation in an amount sufficient to reduce the concentration of bacteria by at least 3 logs reduction at the fourteenth day after an initial challenge with said bacteria, and to reduce the concentration of bacteria by at least 3 logs reduction at the twenty-eighth day after rechallenge with said bacteria at the fourteenth day, wherein the initial challenge is carried out after the formulation has been in storage for 12 months. In one embodiment, the preservative efficacy is demonstrated by the initial challenge that is carried out after the formulation has been in storage for 18 or 24 months.

In still another aspect, a formulation of the present invention is instilled into an affected eye at a dosage of one, two, three, four, or more drops per day, or as prescribed by a skilled medical practitioner. For example, one, two, or three drops of a formulation of the present invention are instilled into an affected eye once, twice, three or more times per day.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A pharmaceutical formulation comprising at least a polyanionic material and at least a source of hydrogen peroxide, which provides an amount of hydrogen peroxide in the formulation in a range from greater than about 0.01 to about 1 percent by weight of the formulation.

2. The pharmaceutical formulation of claim 1, wherein said at least a polyanionic material is selected from the group consisting of alginic acid, carboxymethyl cellulose, carboxymethyl starch, carboxymethyl dextran, dextran sulfate, carboxymethyl chitosan, hyaluronic acid, chondroitin sulfate, xanthan gum, physiologically acceptable salts thereof, derivatives thereof, combinations thereof, and mixtures thereof.

3. The pharmaceutical formulation of claim 2, wherein said at least a polyanionic material is present in an amount in a range from about 0.01 to about 10 percent by weight of the formulation.

4. The pharmaceutical formulation of claim 2, wherein said at least a polyanionic material is present in an amount in a range from about 0.01 to about 5 percent by weight of the formulation.

5. The pharmaceutical formulation of claim 1, wherein said at least a polyanionic material is selected from the group consisting of alginic acid, carboxymethyl starch, carboxymethyl dextran, carboxymethyl chitosan, chondroitin sulfate, physiologically acceptable salts thereof, derivatives thereof, combinations thereof, and mixtures thereof, and said at least a polyanionic material is present in an amount in a range from about 0.01 to about 5 percent by weight of the formulation.

6. The pharmaceutical formulation of claim 1, wherein said at least a source of hydrogen peroxide is selected from the group consisting of hydrogen peroxide, urea hydrogen peroxide, perborate salts, derivatives thereof, and mixtures thereof.

7. The pharmaceutical formulation of claim 6, wherein said at least a source of hydrogen peroxide provides an amount of hydrogen peroxide in the formulation in a range from greater than 0.01 to about 0.5 percent by weight of the formulation.

8. The pharmaceutical formulation of claim 6, wherein said at least a source of hydrogen peroxide provides an amount of hydrogen peroxide in the formulation in a range from greater than 0.1 to about 1 percent by weight of the formulation.

9. The pharmaceutical formulation of claim 2, further comprising a therapeutic agent.

10. The pharmaceutical formulation of claim 9, wherein said at least a source of hydrogen peroxide provides an amount of hydrogen peroxide in the formulation in a range from greater than 0.01 to about 0.5 percent by weight of the formulation.

11. The pharmaceutical formulation of claim 10, wherein the pharmaceutical formulation is an ophthalmic solution, and the therapeutic agent is selected from the group consisting of anti-inflammatory agents, antibiotics, immunosuppressive agents, antiviral agents, antifungal agents, antiprotozoal agents, anti-allergic agents, combinations thereof, and mixtures thereof.

12. The pharmaceutical formulation of claim 11, further comprising a material selected from the group consisting of buffers, tonicity adjusting agents, surfactants, viscosity adjusting agents, combinations thereof, and mixtures thereof.

13. The pharmaceutical formulation of claim 12, wherein the ophthalmic solution provides a medicament for treatment for dry eye, allergy of an eye, inflammation of an eye, or infection of an eye.

14. The pharmaceutical formulation of claim 1, further comprising: a second preservative and a material selected from the group consisting of buffers, tonicity adjusting agents, surfactants, viscosity adjusting agents, antioxidants, vitamins, combinations thereof, and mixtures thereof.

15. The pharmaceutical formulation of claim 1, wherein the pharmaceutical formulation is free of chelating agents.

16. The pharmaceutical formulation of claim 15, wherein said at least a polyanionic material is selected from the group consisting of alginic acid, carboxymethyl cellulose, carboxymethyl starch, carboxymethyl dextran, dextran sulfate, carboxymethyl chitosan, chondroitin sulfate, xanthan gum, physiologically acceptable salts thereof, derivatives thereof, combinations thereof, and mixtures thereof.

17. The pharmaceutical formulation of claim 1, further comprising a second preservative, wherein a concentration of the source of hydrogen peroxide provides hydrogen peroxide at a concentration in a range from about 0.0001% to less than about 0.1% by weight of the total formulation.

18. The pharmaceutical formulation of claim 24, wherein the pharmaceutical formulation comprises a contact-lens care solution.

19. An ophthalmic formulation comprising: boric acid, at least a polyanionic material, and at least a source of hydrogen peroxide, which provides an amount of hydrogen peroxide in the formulation in a range from greater than about 0.01 to about 1 percent by weight of the formulation.

20. The ophthalmic formulation of claim 19, further comprising a stabilized oxychloro complex in an amount from 0.0001 to about 0.01% by weight of the total formulation.

21. A method for making a pharmaceutical formulation, the method comprising: (a) providing an initial formulation; and (b) adding at least a polyanionic material and at least a source of hydrogen peroxide to the initial formulation to produce the pharmaceutical formulation; wherein said at least a source of hydrogen peroxide provides an amount of hydrogen peroxide in the formulation in a range from greater than about 0.1 to about 1 percent by weight of the formulation.

22. The method of claim 21, further comprising adding another ingredient selected from the group consisting of therapeutic agents, buffers, tonicity adjusting agents, surfactants, viscosity adjusting agents, antioxidants, combinations thereof, and mixtures thereof to the pharmaceutical formulation.

23. The method of claim 22, wherein therapeutic agents can be selected from the group of anti-inflammatory agents, antibiotics, immunosuppressive agents, antiviral agents, antifungal agents, and antiprotozoal agents.

24. The method of claim 21, further comprising adding boric acid to the formulation.

25. A method for providing safety, or comfort, or both to users of a pharmaceutical formulation, the method comprising adding at least a polyanionic material and at least a source of hydrogen peroxide to the pharmaceutical formulation, wherein said at least a source of hydrogen peroxide provides an amount of hydrogen peroxide in the formulation in a range from greater than about 0.01 to about 1 percent by weight of the formulation.

26. The method of claim 25, wherein said at least a source of hydrogen peroxide provides an amount of hydrogen peroxide in the formulation in a range from greater than about 0.1 to about 1 percent by weight of the formulation.

27. The method of claim 25, wherein said at least a source of hydrogen peroxide provides an amount of hydrogen peroxide in the formulation in a range from greater than about 0.001 to about 0.1 percent by weight of the formulation, and said formulation further comprises a second preservative.

28. The method of claim 25, wherein the polyanionic material is selected from the group consisting of alginic acid, carboxymethyl cellulose, carboxymethyl starch, carboxymethyl dextran, dextran sulfate, carboxymethyl chitosan, hyaluronic acid, chondroitin sulfate, xanthan gum, physiologically acceptable salts thereof, derivatives thereof, combinations thereof, and mixtures thereof.

29. The method of claim 26, wherein the polyanionic material is selected from the group consisting of alginic acid, carboxymethyl cellulose, carboxymethyl starch, carboxymethyl dextran, physiologically acceptable salts thereof, derivatives thereof, combinations thereof, and mixtures thereof, and the polyanionic material is present in an amount from about 0.01 to about 10 percent by weight of the total formulation.

30. A method for treating or preventing a condition of an eye that manifests irritation or inflammation, the method comprising topically administering to the eye an effective amount of an ophthalmic solution that comprises an ophthalmically active agent, at least a polyanionic material, and at least a source of hydrogen peroxide, which provides an amount of hydrogen peroxide in the formulation in a range from greater than about 0.01 to about 1 percent by weight of the formulation.

31. The method of claim 30, wherein the condition is dry eye condition and the ophthalmically active agent promotes lubrication of an ocular surface.

32. The method of claim 31, wherein a tonicity of the solution is in a range from about 200 to about 270 mOsm/kg.

33. The method of claim 30, wherein the polyanionic material is selected from the group consisting of alginic acid, carboxymethyl cellulose, carboxymethyl starch, carboxymethyl dextran, dextran sulfate, carboxymethyl chitosan, hyaluronic acid, chondroitin sulfate, xanthan gum, physiologically acceptable salts thereof, derivatives thereof, combinations thereof, and mixtures thereof.

34. The method of claim 30, wherein the eye condition is selected from the group consisting of dry eye, inflammation of an eye, infection of an eye, and combinations thereof.

35. A method for treating an ophthalmic device, the method comprising contacting the ophthalmic device with an ophthalmic solution comprising at least a polyanionic material and at least a source of hydrogen peroxide, which provides an amount of hydrogen peroxide in the solution in a range from greater than about 0.1 to about 1 percent by weight of the formulation.

36. The method of claim 35, wherein the ophthalmic solution has the capability to clean, disinfect, and wet or rewet the ophthalmic device.

37. The method of claim 36, wherein the ophthalmic solution further comprises a material selected from the group consisting of surfactants, tonicity adjusting agents, buffering agents, combinations thereof, and mixtures thereof.

38. The method of claim 35, wherein the polyanionic material is selected from the group consisting of alginic acid, carboxymethyl cellulose, carboxymethyl starch, carboxymethyl dextran, dextran sulfate, carboxymethyl chitosan, hyaluronic acid, chondroitin sulfate, xanthan gum, physiologically acceptable salts thereof, derivatives thereof, combinations thereof, and mixtures thereof.
